(19) United States
(12) Patent Application Publication
(10) Pub. No.: US 2002/0012666 A1
(43) Pub. Date: Jan. 31, 2002

(54) A METHOD FOR INCREASING WEIGHT GAIN AND FEED EFFICIENCY

(76) Inventors: HELLEN CHAYA GREENBLATT, WILMINGTON, DE (US); JEFFREY G. HUNCHAR, WEST CHESTER, PA (US); CRAIG W. HENRY, BRUNSWICK, ME (US); SUBRAMANIAN IYER, HOCKESSIN, DE (US); AL D. FARALDO, WEST CHESTER, PA (US)

Correspondence Address: BASIL S KRIKELIS, DCV INC, LEGAL DEPARTMENT, 3521 SILVERSIDE ROAD QUILLEN BUILDING, WILMINGTON, DE, 19810

(21) Appl. No.: 09/414,403

(22) Filed: Oct. 7, 1999

Related U.S. Application Data
(60) Provisional application No. 60/107,128, filed on Nov. 5, 1998.

(51) Int. Cl. A61K 39/395; A61K 39/40; A61K 39/42; A61K 39/44

(52) U.S. Cl. 424/183.1

(57) ABSTRACT

A method for increasing weight gain and feed efficiency in a subject animal, whether that subject animal is healthy or suffering from a disease resulting in weight loss, such as cachexia. The method consists of the administration of an effective amount of hyperimmunized egg product.

### RELATED APPLICATIONS

This application claims the benefit of U.S. Provisional Application No. 60/107,128 filed on Nov. 5, 1998.

### FIELD OF THE INVENTION

The invention relates to a method for increasing weight gain and feed efficiency in a subject. More particularly, the invention relates to a composition, and method for using the same, for increasing weight gain for the purpose of treating and preventing certain disorders which result in weight loss, such as cancer or HIV related wasting syndrome or cachexia.

### BACKGROUND OF THE INVENTION

Improved weight gain and feed efficiency is a major goal of those involved in growing food producing animals. The major advantage of improved weight gain and feed efficiency is that market weight of food animals can be achieved in less time, using less feed, resulting in reduced cost of production.

Attempts to increase weight gain and feed efficiency have been made with the use of antibiotics, steroids and growth hormones. All of these treatments while somewhat effective, can have problems associated with use, such as development of resistance to antibiotics, sterility and gastrointestinal-related disorders. In addition, increased public concern over the use of antibiotics has led to a ban of these products in certain countries.

As such, there is a need for an effective mode of increasing weight gain and feed efficiency in production animals without the resulting side effects associated with present treatments.

Weight gain is also often an essential treatment for certain diseases suffered by animals and humans alike. In particular, wasting syndrome, also known as cachexia, is a devastating disease characterized by progressive weight loss, weakness, fever and diarrhea. In addition, cachexia is often associated with severe loss of body fat, increased lipodysis, and increased turnover of free fatty acid and glycerol. This disease arises as a complication in, for example, persons suffering from advanced human immunodeficiency virus (HIV) infection or AIDS (Acquired Immune Deficiency Syndrome), cancer, the geriatric population, those having diarrhea, and patients undergoing treatment by chemotherapy or radiotherapy. When cachexia is accompanied by malnutrition, as is often the case, the frequency and severity of the disease is generally associated with adverse clinical outcomes. For instance, although a suffering subject may ingest sufficient calories, a majority of these wasting syndrome subjects lose vital nutrients due to diarrhea, vomiting or malabsorption. The effects of wasting syndrome result in diminishing the quality of life, increasing susceptibility to opportunistic infections, exacerbating illness, and increasing the risk of death in those suffering from the disease.

As alluded to earlier, one major concern of wasting syndrome is the weight loss, or lack of weight gain that is associated with it. Some factors, aside from infection, that might be involved in the etiology of weight loss are: decreased caloric intake, malabsorption of nutrients, altered energy utilization or expenditure secondary to infection, and hormonal and metabolic abnormalities.

HIV is one of the major diseases resulting in cachexia, since the gastrointestinal tract is a primary target for AIDS related diseases. Alimentary impairment associated with wasting syndrome results from decreased intestinal surface area, defects in the mucosal function, or rapid cell turnover producing immature epithelium. Mortality from wasting syndrome is directly related to the extent of tissue depletion, and, as such, restoration of body cell mass can enhance survival.

The occurrence of wasting syndrome in HIV subjects can occur as a result of the HIV infection itself or HIV-related opportunistic infections. In the specific case of HIV-related wasting syndrome, gastrointestinal infection may be caused by secondary infection(s) to a diverse collection of enteric pathogens. These pathogens include Cytomegalovirus, Candida, Cryptococcus, Cryptosporidium, Isospora, Shigella, Salmonella and Mycobacterium avium-intracellularae (refs). Partial villus atrophy, crypt hyperplasia, or increased numbers of intraepithelial lymphocytes suggest microbial or immune mediated intestinal damage may be important factors in AIDS enteropathies and malabsorption intestinal injury or damage from protozoal, parasitic, bacterial, or viral infections can result in significant functional impairment. There is also evidence of perturbations in lipid metabolism, and energy/nitrogen balance in the AIDS wasting syndrome as well as suggestions of gastroenterologic dysfunction from infection or inflammatory cytokines.

Cachexia is also associated with cancer patients. Cancer cachexia is a major factor influencing quality of life, survival and outcome of human cancer patients undergoing chemotherapy, radiotherapy or surgical treatment. Human patients who are cachectic often have a lower frequency of tumor regression after chemotherapy and have increased morbidity and mortality during and after surgery. Also, survival in human patients who have not lost weight is prolonged, often up to 50% longer, after treatment for cancer, compared to similarly treated human patients who had lost weight at the time of initial examination. Mean duration for hospital stay of malnourished human cancer patients is twice as long as that of diagnosis-adjusted, well-nourished human patients. Also, weight loss in human cancer patients correlates to amount of activity (performance status) and, therefore, to quality of life.

Cytokines, such as interleukin- 1 (IL1β), tumor necrosis factor (TNF-α), and interferon (IFN) have been implicated in the pathophysiology of cachexia. For example, the cytokine TNF-α is a major inflammatory response mediator implicated in cachexia, rheumatoid arthritis, Crohn's disease, and other autoimmune conditions. Evidence suggests that TNF α is involved in protein mobilization of muscle mass, and is therefore responsible for the metabolic changes associated with cachexia. Tristetraprolin-deficient mice develop autoimmune conditions including cachexia wherein administration of anti-TNF-α antibodies can prevent these conditions. Also, neutralizing antibodies to TNF-α or their receptors have demonstrated efficacy in clinical trails.

In certain cases, while the weight loss resulting from cachexia in a subject can be more than 10 percent of the subject's body weight, such weight loss can be reversed by treating the particular underlying infection. The majority of subjects, however, do not respond to conventional regimens for treatment and prevention of the underlying infection. In addition to treatment of the underlying infection, several other approaches of limited success have been used to treat the effects of wasting syndrome, including appetite stimuli, anabolic agents, cytokine inhibitors, and hormones. Goals of these treatments are to not only increase body weight but also to increase lean body mass and muscle. The development of a successful strategy to increase the body weight and lean muscle mass of individuals with wasting syndrome is critical because decreases in lean body mass are associated with decreased survival of these individuals. The progress of this syndrome begins with depletion of both body fat-free and fat mass followed by an increase in resting energy expenditure. Although the progress of wasting syndrome is accelerated in the presence of infection, few of these therapies have proven successful in stabilizing and/or increasing the weight of patients with wasting syndrome.

As such, there is a true need for a method of increasing weight gain in those suffering from cachexia which is applicable to any form of cachexia. In other words, a method for increasing weight gain that is not specific to an underlying infection or disease.

The present invention provides a novel for increasing weight gain and feed efficiency in both healthy and sick individuals. This method comprises the administration of hyperimmune egg to a subject in need of gaining weight. The prior art does not disclose or suggest that hyperimmune eggs can be administered to subjects in order to increase weight gain and feed efficiency and/or treat wasting syndrome. Nor does the prior art disclose or suggest a method providing a reasonable expectation that hyperimmunization of an avian with a non-specific vaccine could produce an avian which lays eggs having such a capability.

### SUMMARY OF THE INVENTION

In one aspect of the present, the ingestion of the hyperimmune egg product provides a statistically significant increase in weight gain and feed efficiency in animals.

Another aspect of the present invention, the ingestion of hyperimmune egg product provides a significant increase in the weight in many subjects suffering from cachexia.

In still another aspect, the hyperimmune egg product provides a general improvement in the overall health, well being and quality of life of subjects suffering from cachexia. In yet another aspect, in subjects with severe diarrhea and malaise and even subjects that are terminally ill, the hyperimmune egg product, in beverage form, was found to be the only food that these subjects were able to consume.

### BRIEF DESCRIPTION OF THE DRAWING

FIG. 1 is a pie chart showing the percent weight differences in subjects with wasting disease after one month on hyperimmune egg product.

FIG. 2 is a pie chart showing the percent of weight differences in subjects with wasting disease after two months on hyperimmune egg product.

### DESCRIPTION OF THE INVENTION

### Definitions

The terms “egg” or “egg product” each mean any whole egg (table, hyperimmunized or otherwise) or any product or fraction derived therefrom.

The terms “table egg” or “table egg product” each mean a whole egg, or any product or fraction derived therefrom, obtained from egg-producing animals which are not maintained in a hyperimmune state.

The terms “hyperimmune egg” or “hyperimmune egg product” each mean whole egg or any product or fraction derived therefrom, obtained from an egg producing animal maintained in a hyperimmune state.

The term “supranormal levels” means levels in excess of those found in eggs of egg-producing animals not maintained in a hyperimmune state.

The term “immunogen” means a substance that is able to induce a humoral antibody and/or cell-mediated immune response and react with the products of it, e.g., antibody.

The term “immunomodulator” means a substance, other than an antibody, that affects the immune system.

The term “combinatorial derived immunogens” refers to a novel process of generating molecular diversity among immunogens by way of combinatorial synthesis.

The term “bioengineered immunogens” refers to immunogens which are obtained through the process of gene cloning technologies and genetic manipulation which allow the insertion of encoding nucleotides which can give rise to epitopes having immunogenic properties.

The term “genetic vaccine” refers to a nucleic acid vaccine which is generally produced by recombinant technologies and which may elicit an immune response.

The terms “wasting syndrome”, “wasting disease” or “cachexia” are used synonymously and are defined in the background portion of this document.

The term “treatment” means that the onset of the symptoms of the disorder and/or pathogenic origin of the disorder be delayed or completely prevented, or, if present, the symptoms be ameliorated or completely eliminated.

The term “prevention” means that the progression of the disease is reduced and/or eliminated, or that the onset of the disease is eliminated.

The term “administer” means any method of providing a subject with a substance, including orally, intranasally, intraoptically, parenterally (intravenously, intramuscularly, or subcutaneously), rectally or topically.

The term “animal” means the animal kingdom definition.

The term “target animal” refers to an animal which functions as the egg or egg product producing animal.

The term “subject animal” refers to the animal which is administered the egg or egg product produced by the target animal.

The terms “feed efficiency” or “feed conversion” expresses the efficiency by which an animal converts feed into weight gain. Feed efficiency is expressed as the ratio of weight of feed to weight gain.

The term “weight adjusted feed efficiency” or “weight adjusted feed conversion” is based upon a feed efficiency corrected to a standard weight called the “weight adjusted feed efficiency” (WAFE). Feed efficiency can be adjusted to a standard weight of 2.00 kg. For every 31.78 grams difference in weight, the feed efficiency can be adjusted by 0.01. For example:

a) If the actual weight is higher than the standard weight then the feed efficiency is adjusted downward. If the actual weight is lower than the standard weight then the feed efficiency is adjusted upward. For example:

b) Assume a treatment group has an average weight of 2.032 kg with a 2.0 feed efficiency.

c) Based upon an efficiency factor of 31.78 grams of weight equaling 0.01 kg of feed efficiency then the WAFE would equal 1.99 for a standard of 200 kg.

The terms “increased weight” or “increased weight gain” means an increase in weight.

The term “weight loss” means a decrease in weight.

### The Invention

The present invention uses a novel approach for increasing weight gain and feed efficiency. In a preferred embodiment, the present invention uses this approach to ameliorate, treat and/or prevent wasting syndrome in subjects suffering from the disease. The invention comprises a hyperimmune egg product containing antibodies to common human enteric pathogens and other immune components in addition to antibodies. Upon ingestion of a predetermined amount of the hyperimmune egg product, subjects showed a general weight gain of 0.5-6 kg of body weight and a significant reduction or complete elimination of wasting syndrome related symptoms (See Example 2).

### Mechanism of Action

The mechanism of action of the hyperimmune egg for increasing weight gain may be due to any of the following: directly or indirectly contributing to pathways that maintain a healthy turnover of cells; regulating the cytokines involved in modulating the immune system and thus supporting a healthier g. i. tract; changing the ratio of cytokines or other immunoregulatory factors that lead to Wasting Disease; “directly coating” the g.i. tract and/or; lowering the level of gastrointestinal flora.

The inventors suggest that one specific mode by which these subjects may be benefiting by the ingestion of the hyperimmune egg product is by reduction of gastrointestinal bioburdens by antibodies that are present in the hyperimmune egg product. Subjects with reported improvements in their weight and skeletal mass may have had their subclinical infection rates lowered, thus optimizing their health and permitting more energy to go toward growth rather than suppression of opportunistic organisms. However, although this may be one mode of action of the hyperimmune egg, this mode is particular only to those with infections. Example 3 shows increased weight gain and feed efficiency in perfectly healthy subjects not suffering from any particular infection.

Another mode by which the hyperimmune egg increases weight gain and feed efficiency may be due to the effect of immunomodulators within the egg. For example, the activities of the hyperimmune egg as it relates to immune regulation is consistent with a model suggesting that the immunomodulators found in the egg are able to regulate the production of immune factors directly and thereby stimulate the immune system in the cells lining the gastrointestinal tract and modulate processes that lead to cachexia.

### Preparation Of Hyperimmune Egg Product

It is important to keep in mind that the hyperimmunization process can be performed with either avians, whereby the egg contains the beneficial elements resulting from the hyperimmunization process, or with bovine whereby the milk contains the beneficial elements resulting from the hyperimmunization process. The below description is limited to hyperimmunization of avians, although the same concept is applicable to the hyperimmunization of bovine and the collection of their hyperimmune milk.

The hyperimmune egg product can be produced by any egg-producing animal. It is preferred that the animal be a member of the class Aves or, in other words, an avian. Within the class Aves, domesticated fowl are preferred, but other members of this class, such as turkeys, ducks, and geese, are a suitable source of hyperimmune egg product.

The hyperimmune egg product is provided as a spray dried egg powder and is obtained from laying hens vaccinated with a panel of human enteric pathogens (see Example 1). It is submitted that any immunogen or collection of immunogens can be used in the hyperimmunization process of this invention. The process of spray drying the pasteurized liquid egg minimizes damage to the antibodies and immune modulators in the egg, resulting in a product that has a high nutrient value and is capable of conferring passive protection to opportunistic enteric infections and appears capable of decreasing inflammation. Antibodies, as a group, are especially resistant to destruction by normal enzymes, and upon oral consumption, a significant fraction will pass through the gastrointestinal tract intact and active. Numerous studies report that orally consumed antibodies offer protection against specific enteric agents.

Further, when such egg-producing animals are brought to a specific state of immunization by means of, for example, periodic booster administrations of antigens, the animals will produce eggs that, when consumed by a subject, will have beneficial properties which cause increases in weight gain and/or feed efficiency in a subject.

Having knowledge of the requirement for developing and maintaining a hyperimmune state, it is within the skill of the art to vary the amount of immunogen administered, depending on the egg-producing animal genera and strain employed, in order to maintain the animal in the hyperimmune state.

Alternative modes of hyperimmunizing egg producing animals can be used which, in place of immunogenic vaccines, include the use of genetic vaccines. In particular, any DNA construct (generally consisting of a promoter region and an immunogen encoding sequence) will trigger an immune response. Genetic vaccines consist of immunogen-coding vectors, fragments of naked DNA, plasmid DNA, DNA-RNA antigens, DNA-protein conjugates, DNA-liposome conjugates, DNA expression libraries, and viral and bacterial DNA delivered to produce an immune response. Methods of DNA delivery include particle bombardment, direct injection, viral vectors, liposomes and jet injection, among others. When applying these delivery methods, much smaller quantities may be necessary and generally result in more persistent immunogen production. When using such genetic processes, the preferred method for introducing DNA into avians is through intramuscular injection of the DNA into the breast muscle.

### Preferred Hyperimmunization Procedure

The following list of steps is an example of a preferred procedure used to bring an egg-producing animal to a heightened state of immunity:

1. Selecting one or more immunogens.

2. Eliciting an immune response in the egg-producing animal by primary immunization.

3. Administering booster vaccines of immunogens of appropriate dosage to induce and maintain the hyperimmune state.

Step 1: Any immunogens or combination of immunogens may be employed as a vaccine. The immunogens can be bacterial, viral, protozoan, fungal, cellular, or any other substances to which the immune system of an egg-producing animal will respond. The critical point in this step is that the immunogen(s) must be capable of inducing immune and hyperimmune states in the egg-producing animal. Although only a single antigen may function as the vaccine for the method of the invention, one preferred vaccine is a mixture of polyvalent bacterial and viral antigens selected from the following antigen families: the enteric bacilli and bacteroides, Pneumococci, Pseudomonas, Salmonella, Streptococci, Bacilli, Staphylococci, Neisseria, Clostridia, Mycobacteria, Actinomycetes Chlamydiae, and Mycoplasma. Viral antigens are preferably selected from the following antigen families: adenoviruses, picornaviruses and herpes viruses, although other viral antigen families will work.

In an alternative embodiment, a polyvalent vaccine referred to as PL 100 ( is used.

The bacteria included in the PL-100 vaccine are listed in table 1 of Example 1.

This vaccine has been previously described in U.S. Pat. Nos. 5,106,618 and 5,215,746, (described as S-100), both assigned to Stolle Research and Development Corporation.

Step 2: The vaccine can be either a killed or live-attenuated vaccine and can be administered by any method that elicits an immune response. It is preferred that immunization be accomplished by administering the immunogens through intramuscular injection. The preferred muscle for injection in an avian is the breast muscle. Other methods of administration that can be used include intravenous injection, intraperitoneal injection, intradermal, rectal suppository, aerosol or oral administration. When DNA techniques are used for the hyperimmunization process, much smaller quantities are required, generally 1 -100 micrograms.

It can be determined whether the vaccine has elicited an immune response in the egg-producing animal through a number of methods known to those having skill in the art of immunology. Examples of these include enzyme-linked immunosorbent assays (ELISA), tests for the presence of antibodies to the stimulating antigens, and tests designed to evaluate the ability of immune cells from the host to respond to the antigen. The minimum dosage of immunogen necessary to induce an immune response depends on the vaccination procedure used, including the type of adjuvants and formulation of immunogen(s) used as well as the type of egg-producing animal used as the host.

Step 3: The hyperimmune state is preferably induced and maintained in the target animal by repeated booster administrations of an appropriate dosage at fixed time intervals. The time intervals are preferably 2-8 week intervals over a period of 6-12 months. Dosage is preferably 0.05-5 milligrams of the immunogenic vaccine. However, it is essential that the booster administrations do not lead to immune tolerance. Such processes are well known in the art.

It is possible to use other hyperimmunization maintenance procedures or combination of procedures, such as, for example, intramuscular injection for primary immunization and intravenous injection for booster injections. Further procedures include simultaneously administering microencapsulated and liquid immunogen, or intramuscular injection for primary immunization, and booster dosages by oral administration or parenteral administration by microencapsulation means. Several combinations of primary and hyperimmunization are known to those skilled in the art.

### Processing and Administration Of Hyperimmune Egg

Once the egg-producing animals have been sufficiently hyperimmunized, it is preferred that the eggs from these animals are collected and processed to produce a hyperimmune egg product. Subsequently, the hyperimmune egg product can be administered to the subject.

The egg and/or egg product of the present invention is administered to a subject animal by any means that increases weight gain and/or feed efficiency in the subject. It is preferred that administration occur by directly feeding the egg or any effective derivative of the egg. Egg and egg yolk are natural food ingredients and are non-toxic and safe.

One preferred method for preparing the egg involves drying the egg into an egg powder. Although various methods are known for drying eggs, spray drying is a preferred method. The process of spray drying eggs is well known in the art.

In a preferred embodiment, the hyperimmune egg is administered together with a food product containing several nutrients such as vitamins and minerals. Such nutrient-bearing foods are often in the form of a nutritional or dietary supplement. Along these lines, dried egg powder can also be incorporated into drinks in the form of, for example, protein powders, power building drinks, protein supplements and any other nutritional, athlete-associated products. In Example 2, a nutritionally balanced drink was administered together with the hyperimmune egg product, and this drink supported immune function, as the vitamins, minerals, and amino acids in the product enhanced the nutrient intake of the person consuming it.

Nutritional foods play a key role in boosting caloric intake. For instance, in Example 2, besides providing essential vitamins, minerals, and 110 calories of energy, each serving of the nutritional drink provided 11.0 gr of protein, 9 gr of carbohydrates and 4.5 g of fat which may have contributed to the subjects'health.

Therefore, in one embodiment, extra nutrients can be provided to a subject by administering the hyperimmunized egg product in combination with a nutrient bearing carrier, such as a dietary supplement.

In an alternative embodiment, the egg powder can be used in bake mixes, power bars, candies, cookies, etc. Other examples of egg processing include making an omelet, soft or hard-boiling the egg, baking the egg, or, if desired, the egg can be eaten raw or processed as liquid egg.

Finally, it is generally known in the art that the yolk and/or white fractions contain the agent or agents responsible for the beneficial properties observed and referred to above. Those having ordinary skill in the art would clearly recognize that further separation could provide more potent fractions or elimination of undesirable components, and would allow for other modes of administration such as administering egg product parenterally, subcutaneously, intravenously, intramuscularly, intraperitoneally, intranasally, orally or topically. Such further separation will provide for the ability to make encapsulated products and pharmaceutical compositions with said egg or fraction thereof.

When it comes to the treatment and prevention of wasting syndrome, the hyperimmune egg product is preferably administered to the subject in an amount that is immunologically effective in treating and preventing this disorder by stopping weight loss and actually allowing subjects to gain weight.

It is the inventors'finding that administration of anywhere from 100 mg to 10 g of egg per kilogram of subject weight is effective in increasing weight gain and feed efficiency. Duration and intensity of the treatment will depend upon the particular stage of the condition, whether it is present, and, if so, the advancement of the condition in the subject. The hyperimmune egg product is provided in any amount that treats and/or prevents the condition and the symptoms of the condition. For example, in some cases, daily amounts ranging from less than one to several whole, hyperimmune eggs (or hyperimmune egg products containing the equivalent of less than one to several whole, hyperimmune eggs) can be administered to the subject depending on the particular circumstance of the condition. More potent fractions can be separated and concentrated by methods well-known in the art, from several hundred eggs.

The advantageous properties of this invention can be observed by reference to the following examples which illustrate the invention.

### EXAMPLES

### Example 1

### Preparation of PL-100 Vaccine

A bacterial culture containing the spectrum of bacteria shown in Table 1 below, as obtained from the American Type Culture Collection, was reconstituted with 15 mL of media and incubated overnight at 37 C. Once good growth was obtained, approximately one-half of the bacterial suspension was employed to inoculate one liter of broth with the inoculate being incubated at 37 C.

After good growth was visible in the culture, the bacterial cells were harvested by centrifugation of the suspension for 20 minutes to remove the media. The bacterial pellet obtained was resuspended in sterile saline solution and the bacterial sample was centrifuged three times to wash the media from the cells. After the third sterile saline wash, the bacterial pellet was resuspended in a small amount of double distilled water.

The media-free bacterial suspension was killed by placing the suspension in a glass flask in an 80 C. water bath overnight. The viability if the broth culture was tested with a small amount of killed bacteria, incubated at 37 C. for five days and checked daily for growth to certify that the bacteria had been killed.

The killed bacteria were lyophilized until dry. The dry bacteria were then mixed with sterile saline solution to a concentration of 2.2×10⁸ bacterial cells/mL saline (1.0 optical density reading at 660 nm). Bacteria contained in S-100 vaccine are listed in Table 1 below.

TABLE 1
PL-100 Bacterial List

| | |
|---|---|
| Escherichia coli | Escherichia coli (Aerobacter) |
| Klebsiella pneumoniae | Pseudomonas aeruginosa |
| Salmonella typhimurium | Salmonella dysenteriae |
| Salmonella enteriditis | Salmonella epidermis |
| Salmonella simulans | Streptococcus pyogenes, type 1 |
| Streptococcus pyogenes, type 3 | Streptococcus pyogenes, type 5 |
| Streptococcus pyogenes, type 8 | Streptococcus pyogenes, type 12 |
| Streptococcus pyogenes, type 14 | Streptococcus pyogenes, type 18 |
| Streptococcus pyogenes, type 22 | Pseudomonas vulgaris |
| Streptococcus agalactiae | Streptococcus mitis |
| Streptococcus mutans | Streptococcus salavarius |
| Streptococcus sanguis | Streptococcus pneumoniae |
| Propionibacterium acnes | Haemophilis influenzae |

### Immunization Procedure for Hyperimmunized Egg Product

A killed preparation of pathogens was prepared as described above. For the first vaccination, the bacteria were mixed with complete Freund's adjuvant, and 5.6 mg of bacterial material was injected into the breast muscle of a chicken. For the remaining vaccines, the bacterial preparation was mixed with incomplete Freund's adjuvant and injected into the chickens at two-week intervals for six months.

### Example 2

### Weight Gain In Patients Suffering From Cachexia

This study reports on a human clinical trial that was conducted to determine if a dietary supplement containing hyperimmune egg would be able to stop weight loss and increase weight in human patients suffering from wasting syndrome. The duration of the study was 8 weeks, during which the physician evaluated the subject biweekly.

### MATERIALS AND METHODS

### Subjects

Fourteen HIV positive male subjects (as documented by ELISA and Western Blot analysis) ranging in age from 18-50 years and suffering from wasting syndrome were recruited from the Jamshedji Jeejiboy (J.J.) Hospital, Mumbai, India. These individuals had been previously diagnosed with one or more of the AIDS defining secondary conditions.

None had participated in any investigational drug tests within the last 60 days or had been exposed to any immunomodulator or vaccine for the past 90 days.

Subjects who had frequent changes in dosing or types of medication to control clinical symptoms, alcohol or substance abusers, history of allergy towards eggs or any other ingredient in the test article were excluded from the study.

### Study Design

The open-label study testing of a dietary supplement fortified with hyperimmune spray dried egg powder (DCV, Inc. Wilmington, Del. U.S.A.) was conducted for a total of 12 weeks. The dietary supplement beverage, which contained 6 g of hyperimmune egg per serving, was freshly prepared and consumed as a liquid once a day for 8 weeks. At the end of this 2-month period, test article was no longer consumed for the rest of the 1 month duration of the study. The same physician monitored subjects at 4 wk intervals for the entire length of the study.

All subjects maintained their normal diets and medication and were monitored during the 60-day trial period and 30 days thereafter for any untoward signs or symptoms. The severity, onset date, duration, frequency, study product relationship, action taken and outcome of each adverse experiences were recorded

### Global Assessment Of The Subjects

All subjects were required to visit the physician prior to starting the study (baseline visit) and thereafter at 4 wk intervals for a total of four visits. During each visit, the subject received a detailed physical examination and general evaluation of their well being with a list of questions that included a) estimation of frequency of bowel movements, b) consistency of bowel movements, c) number of bouts of nausea, d) differences in general energy levels and e) differences in general feelings of well-being.

### Subjects Response To Therapy

Each subject's perceived well-being was assessed at the end of the study by using eight linear analogue scale questions on the overall treatment effect. The parameters included were: change in quality of life, personal appearance, weight, and appetite (Table 1). Quality of life indexes were compared before and after treatment by use of a physician-applied health survey questionnaire. The questions included emotional well being, physical function, energy/fatigue, general health, pain and social functioning.

### Results

Thirteen of the Fourteen subjects completed the first 30 days of the protocol. 60-day data were available from seven of the fourteen subjects. Two specific case reports are presented in this document as follows:

### CASE #1

Subject #1 was admitted with a weight of 46kg. He had complaints of gripping abdominal pain, nausea, vomiting, diarrhea and was being treated with antispasmodics/antidiarrheal medicines and administration of I.V. fluids.

During the 8 wk trial period, the subject reported reduction in his bouts of nausea and diarrhea. The general well being of the subject appeared improved and he experienced a weight gain from 46 kg to 47.5 kg. (Table 1)

### CASE #2

Subject #2 was admitted with a weight of 53 kg. He complained of respiratory distress and abdominal pain and was treated for Koch's infection. Subject had tested HIV positive for last 6 years and had taken sporadic doses of ayurvedic medicines for chest congestion and cough. Subject was also provided antibiotics, steam inhalations and cough syrup.

After eight weeks of consuming the test article, he showed improvement in health and general well being along with a weight gain of 3 kg. His attacks of abdominal pain and respiratory distress were reduced.

TABLE 1
Global Assessments of Case #1

| | Mid-arm | Mid-thigh | Abdominal | | |
|---|---|---|---|---|---|
| Date | Circumference | Circumference | Girth | Chest | Weight |
| Day 1 | 8.5″ | 13.5″ | 27″ | 31″ | 46 kg |
| Day 57 | 9″ | 13″ | 26″ | 32.5″ | 47.5 kg |

TABLE 2
Global Assessment of Case #2

| | Mid-arm | Mid-thigh | Abdominal | | |
|---|---|---|---|---|---|
| Date | Circumference | Circumference | Girth | Chest | Weight |
| Day 1 | 9.5″ | 15.5″ | 31″ | 31.5″ | 53 kg |
| Day 57 | 9″ | 17″ | 33″ | 32″ | 56 kg |

### Responses Of Subjects

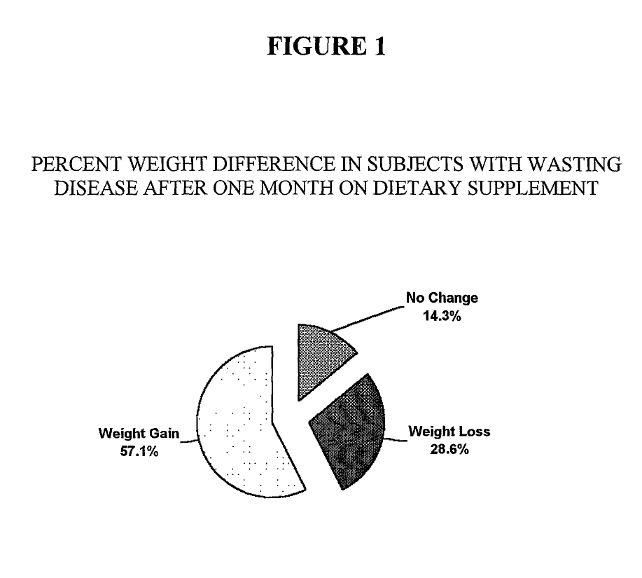

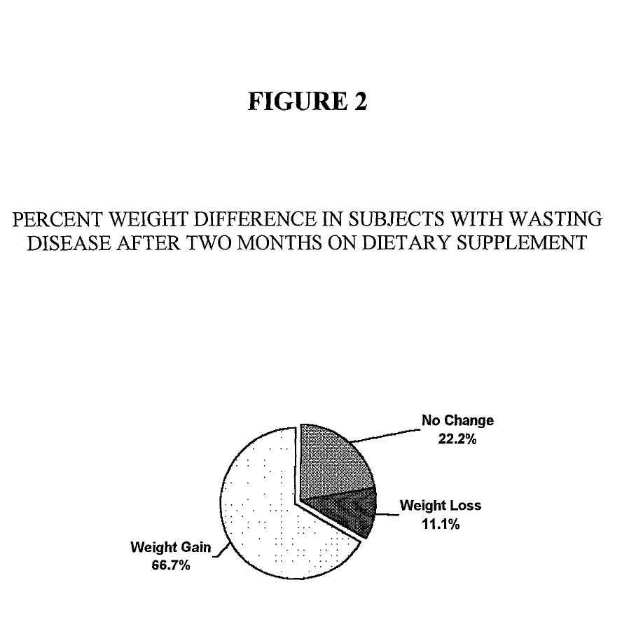

Of the 14 subjects enrolled in the study, 57.1% -66.7% of the subjects gained weight depending on the number of weeks on product (see FIGS. 1 and 2). Weight gains ranged from 0.5 kg to 4 kg. In the same time period weight losses ranged from 11.1% to 28.6% (FIGS. 1 and 2) of the subjects, with the greatest individual weight loss being 6.0 kg. Table 3 below shows the actual weights of the patients at each monthly visit during the trial.

TABLE 3
Weight Changes Over A Three Month Period

| | MONTH NUMBER | | | |
|---|---|---|---|---|
| PATIENT | 0 | 1 | 2 | 3 |
| 1 | 33.0 kg | 35.0 kg | | 35.0 kg |
| 2 | 35.0 kg | 35.0 kg | 36.0 kg | 36.0 kg |
| 3 | 36.0 kg | 35.0 kg | | 34.0 kg |
| 4 | 45.0 kg | 46.0 kg | | 48.0 kg |
| 5 | 35.0 kg | 36.0 kg | 36.0 kg | |
| 6 | 71.0 kg | 71.0 kg | 71.0 kg | |
| 7 | 52.0 kg | 52.5 kg | | |
| 8 | 58.0 kg | 60.5 kg | 62.0 kg | 62.0 kg |
| 9 | 46.0 kg | 46.5 kg | 48.0 kg | |
| 10 | 44.5 kg | 42.5 kg | 42.0 kg | |
| 11 | 53.0 kg | 56.0 kg | 56.0 kg | 58.0 kg |
| 12 | 59.0 kg | 58.5 kg | 56.0 kg | 58.0 kg |
| 13 | 51.0 kg | 52.0 kg | 52.5 kg | |
| 14 | 51.0 kg | 45.0 kg | | |

### Quality Of Life

Quality of life indexes as assessed by the survey showed a marked improvement both in the physical and emotional status of the subjects. In all six domains including physical health, emotional well being, pain, energy levels, general health and fatigue, there were marked improvement reported. Many subjects also experienced a major reduction, or complete abolition, of the number of diarrheal episodes. In at least one instance a dying subject consumed only this product as his sole source of nutrition until his death.

### Compliance, Side Effects, And Adverse Events

Compliance was confirmed by registered subject visits and empty pouch counts. One subject died of AIDS-related complications before completing the study. No subject withdrew from the study because of the drink-related side effects.

### EXAMPLE 3

### Weight Gain and Feed Efficiency In Broilers

576 broilers were separated into 12 groups of 72. The broilers were all 1-day-old hatchlings at the start of the experiment, and the total broilers were a straight run of males and females. Hyperimmune egg was added to the feed of the broilers at 100 g perton offeed. This roughly comes out to approximately 0.07 g of hyperimmune egg per broiler per week to a total of approximately 0.1 g of hyperimmune egg per pound of broiler for the lifetime of the broiler. Table 4 below shows the results of the trial.

TABLE 4
Trial, 8 reps/Treatment, 72 Birds/rep, CobbxCobb, 49 days old

| Treat- | | # | % | Live | Wt. | Adj. |
|---|---|---|---|---|---|---|
| ment | Treatment | Birds | Mort. | Wt.(lbs) | Adj. F.C. | F.C. |
| 6 | BMD & Nitro & | 576 | 5.63a | 5.181ab | 1.864a | 1.838 |
| | Ovation(water) & | | | | | |
| | PL100 | | | | | |
| 5 | BMD & Nitro & | 576 | 5.16a | 5.053cd | 1.891bc | 1.883 |
| | Ovation(water) | | | | | |
| 4 | Ovation (water) | 576 | 1.88a | 5.013de | 1.907c | 1.905 |
| 3 | BMD & Nitro | 576 | 4.22a | 4.930ef | 1.906c | 1.916 |
| 2 | Nitro | 576 | 3.13a | 4.834fg | 1.935d | 1.958 |
| 1 | No Additives | 576 | 2.34a | 4.784g | 1.95d | 1.981 |

### Conclusion

The data in this example, as set forth in Table 4 above, shows an increase in weight gain, feed conversion and weight adjusted feed conversion in chickens from age 1 day to age 49 days. The increase is best seen by comparing treatment 6 with treatment 5. The addition of the hyperimmune egg product (PL100) to the diets given in treatment 5 increased weight gain, feed conversion an weight adjusted feed conversion in a statistically significant manner.

The invention has been described with particular reference to certain preferred embodiments thereof, but it will be understood that variations and modifications can be effected within the spirit and scope of the invention as described above and as defined in the appended claims.

1. A method for increasing weight gain in a subject animal comprising administering to said animal an effective amount of an egg product wherein the egg product is obtained from an egg-producing animal which has been hyperimmunized with an immunogenic vaccine, said vaccine comprising at least one immunogen selected from the group consisting of the following bacterial strains:
Escherichia coli; Escherichia coli (Aerobacter); Klebsiella pneumoniae; Pseudomonas aeruginosa; Salmonella typhimurium; Salmonella dysenteriae; Salmonella enteriditis; Salmonella epidermis; Salmonella simulans; Streptococcus pyogenes, type 1; Streptococcus pyogenes, type 3; Streptococcus pyogenes, type 5; Streptococcuspyogenes, type 8; Streptococcuspyogenes, type 12; Streptococcus pyogenes, type 14; Streptococcus pyogenes, type 18; Streptococcus pyogenes, type 22; Pseudomonas vulgaris; Streptococcus agalactiae; Streptococcus mitis; Streptococcus mutans; Streptococcus salavarius; Streptococcus sanguis; Streptococcus pneumoniae; Propionibacterium acnes; and Haemophilis influenzae.

2. The method of claim 1 wherein the egg product is selected from the group consisting of whole egg, egg yolk, egg white and any fraction thereof.

3. The method of claim 1 wherein the effective amount of egg product administered is between approximately 100 milligrams and 10 grams of whole egg per kilogram of subject weight, or the equivalent thereof for a fraction of the whole egg.

4. The method of claim 1 wherein the egg-producing animal is a member of the avian class.

5. The method of claim 4 wherein the egg-producing animal is selected from the group consisting of fowl, turkey, duck, and goose.

6. The method of claim 1 further comprising a method for increasing feed efficiency in the subject animal.

7. The method of claim 6 wherein the egg product is selected from the group consisting of whole egg, egg yolk, egg white and any fraction thereof.

8. The method of claim 6 wherein the effective amount of egg product administered is between approximately 100 milligrams and 10 grams of whole egg per kilogram of subject weight, or the equivalent thereof for a fraction of the whole egg.

9. The method of claim 6 wherein the egg-producing animal is a member of the avian class.

10. The method of claim 9 wherein the egg-producing animal is selected from the group consisting of fowl, turkey, duck, and goose.

11. A method for treating and preventing cachexia in a subject animal comprising administering to the subject animal an effective amount of an egg product.

12. The method of claim 11 wherein the egg product is selected from the group consisting of whole egg, egg yolk, egg white and any fraction thereof.

13. The method of claim 12 wherein the egg product is obtained from an egg-producing animal which has been hyperimmunized with a vaccine.

14. The method of claim 13 wherein the vaccine comprises at least one immunogen selected from the group consisting of bacterial, viral, protozoan, fungal and cellular immunogens and mixtures thereof.

15. The method of claim 13 wherein the vaccine comprises at least one immunogen selected from the group consisting of the following bacterial strains:
Escherichia coli; Escherichia coli (Aerobacter); Klebsiella pneumoniae; Pseudomonas aeruginosa; Salmonella typhimurium; Salmonella dysenteriae; Salmonella enteriditis; Salmonella epidermis; Salmonella simulans; Streptococcus pyogenes, type 1; Streptococcus pyogenes, type 3; Streptococcus pyogenes, type 5 ; Streptococcus pyogenes, type 8; Streptococcus pyogenes, type 12; Streptococcus pyogenes, type 14; Streptococcus pyogenes, type 18; Streptococcus pyogenes, type 22; Pseudomonas vulgaris; Streptococcus agalactiae; Streptococcus mitis; Streptococcus mutans; Streptococcus salavarius; Streptococcus sanguis; Streptococcus pneumoniae; Propionibacterium acnes; and Haemophilis influenzae.

16. A method for improving the overall health, well-being and quality of life of a subject animal comprising administering to the subject animal an effective amount of an egg product.

17. The method of claim 16 wherein the subject animal is suffering from a disease which has the effect of diminishing the overall health, well being and quality of life of the subject animal.

18. The method of claim 17 wherein the disease is cachexia.

19. The method of claim 16 wherein the egg product is obtained from an egg-producing animal which has been hyperimmunized with a vaccine.

20. The method of claim 18 wherein the vaccine comprises at least one immunogen selected from the group consisting of the following bacterial strains:
Escherichia coli; Escherichia coli (Aerobacter); Klebsiella pneumoniae; Pseudomonas aeruginosa; Salmonella typhimurium; Salmonella dysenteriae; Salmonella enteriditis; Salmonella epidermis; Salmonella simulans; Streptococcus pyogenes, type 1; Streptococcus pyogenes, type 3; Streptococcus pyogenes, type 5; Streptococcus pyogenes, type 8; Streptococcus pyogenes, type 12; Streptococcus pyogenes, type 14; Streptococcus pyogenes, type 18; Streptococcus pyogenes, type 22; Pseudomonas vulgaris; Streptococcus agalactiae; Streptococcus mitis; Streptococcus mutans; Streptococcus salavarius; Streptococcus sanguis; Streptococcus pneumoniae; Propionibacterium acnes; and Haemophilis influenzae.
